(19) United States
(12) Patent Application Publication
(10) Pub. No.: US 2021/0213182 A1
(43) Pub. Date: Jul. 15, 2021

(54) MEDICAL HYDROGEL HAVING RADIATION PROTECTION FUNCTION

(71) Applicant: SHANGHAI RUINING BIOTECHNOLOGY CO. LTD, Shanghai (CN)

(72) Inventors: Zhen PAN, Shanghai (CN); Liang CHEN, Shanghai (CN); Sen HOU, Shanghai (CN)

(21) Appl. No.: 17/274,134

(22) PCT Filed: Jul. 4, 2019
(86) PCT No.: PCT/CN2019/094653

§ 371 Date: Mar. 5, 2021

(30) Foreign Application Priority Data
Feb. 2, 2019 (CN) 201910108095.1

(51) Int. Cl. A61L 31/14; A61N 5/10; A61L 31/06; C08J 3/24; C08J 3/075

(52) U.S. Cl. CPC A61L 31/145; A61N 5/10; A61L 31/06; C08J 2371/02; C08J 3/075; A61N 2005/1094; C08J 3/243

(57) ABSTRACT

A medical hydrogel is formed by in-situ crosslinking an aldehyde-terminated multi-arm star polyethylene glycol and a polyamino compound. The aldehyde group and the multi-arm star polyethylene glycol are linked by a chemical bond such as an ether bond, an amide bond, an ester bond, a urethane bond, an imine bond, or a urea bond. The molar ratio of the amino in the polyamino compound to the aldehyde group in the aldehyde-terminated multi-arm star polyethylene glycol is 0.4-4.4:1. The polyamino compound is polylysine or a mixture of polylysine and polyethylenimine in a molar ratio of 2-30:3.

### BACKGROUND

### Technical Field

The present invention relates to the field of biomedical technologies, and specifically to a medical hydrogel, which may be used as a radiation protection material for radiotherapy spacers, postoperative tissue closure and anti-leakage, anti-tissue adhesion, tissue filler, tissue repair, skin dressing and drug releasing.

### Related Art

Hydrogel is a soft material containing a large amount of water that is obtained by crosslinking of hydrophilic polymers. It has good physicochemical properties and biological characteristics, such as high water content, high elasticity, softness, and biocompatibility, and has important application value in the biomedical research fields, such as drug delivery and tissue engineering. Injectable hydrogel is a type of hydrogel with a certain fluidity that can be applied by injection. Under external stimuli (changes in temperature, temperature/pH, etc.), the injectable hydrogel presents a phase transition between sol and gel. It is in a liquid state or in a semi-solid state having shear thinning property before being injected into a human body. After being injected into the human body, the injectable hydrogel can gel in situ, and thus no invasive surgeries are required, thereby effectively avoiding the risks of infection, and relieving the pains of patients. Various injectable PEG hydrogels current developed include amphiphilic polyester/polypeptide hydrogels with PEG as a hydrophilic segment, PEG hydrogels prepared by supramolecular interaction, and PEG hydrogels prepared through mild chemical reactions.

Polyethylene glycol (PEG) is a type of non-ionic polymer. Having good biocompatibility and safety, PEG is a synthetic polymer approved by U.S. Food and Drug Administration (FDA) for clinical use in humans. PEG can be used as a pharmaceutical excipient, and PEG with active terminal functional groups can be used to modify drugs (PEGylation). PEGylation technology has a large number of advantages, particularly in terms of the modification of proteins and polypeptide drugs, such as prolonging circulation time in the body, enhancing biological activity, avoiding proteolysis and decreasing immune responses. By connecting active terminal functional groups, for example, amino, thiol, azide, alkynyl, and aldehyde, PEG conjugates can be prepared to improve the properties of PEG.

Radiotherapy is a common treatment for cancer. However, radiotherapy may cause unwanted radiation injuries to adjacent healthy tissues. The injuries may affect the health and quality of life of patients during radiotherapy, and persist in the following years. In recent years, radiation oncologists have attempted to decrease the risks of radiation injuries to surrounding tissues during radiotherapy by using a “spacing” technology. PEG hydrogel is one of the ideal materials for tumor radiotherapy spacers. Because the radiotherapy usually has a long treatment period, generally more than 1 month, the hydrogel intended for radiotherapy protection should remain stable in the body for a long period of time. In addition, the hydrogen should gel rapidly after being injected into the body, in order to prevent it from infiltrating into other sites of the body.

CN105963792A discloses a medical hydrogel composition comprising a first component and a second component, wherein the first component includes polylysine and polyethylenimine; and the second component includes one or more of four-arm polyethylene glycol-succinimidyl glutarate, four-arm polyethylene glycol-succinimidyl succinate and four-arm polyethylene glycol-succinimidyl carbonate. When in use, the nucleophiles (polylysine and polyethylenimine) of the first component may undergo Michael addition reaction with the electrophiles (one or more of four-arm polyethylene glycol-succinimidyl glutarate, four-arm polyethylene glycol-succinimidyl succinate and four-arm polyethylene glycol-succinimidyl carbonate) of the second component, and thus can rapidly form a gel with an excellent low swelling property. However, the succinimidyl ester-terminated PEG material has a very short half-life in water, and is easy to be hydrolyzed. Therefore, it can only be preserved at room temperature in the form of powder for a long period of time by a special technology, and must be used immediately (generally 1 h) after dissolution, making it inconvenient to use.

CN107693838A discloses a medical injectable gel and a preparation method thereof, wherein an aldehyde-terminated hyperbranched polymer HP-PEG-CHO solution with a concentration of 2-20% (w/v) is mixed with a polyamino compound solution with a concentration of 2-20% (w/v) through a two-component syringe and then sprayed. The aldehyde-terminated hyperbranched polymer is crosslinked with the polyamino compound by reacting the aldehyde group with the amino to form Schiff base, thereby obtaining a medical injectable gel. The aldehyde group in the aldehyde-terminated hyperbranched polymer HP-PEG-CHO is linked to the polymer through a plurality of ester bonds, and thus the long-term stability in an aqueous solution is relatively low. In addition, the hyperbranched polymer has a wide distribution of molecular weight, and may contain high molecular weight polymers, which is not beneficial to be excreted from human bodies.

### SUMMARY

In view of the shortcomings of the prior art, the present invention provides a medical hydrogel for use in radiation protection materials, wherein the hydrogel has a long-term stability in an aqueous solution.

The specific technical solutions provided in the present invention are as follows:

A medical hydrogel is provided, which is formed by in-situ crosslinking an aldehyde-terminated multi-arm star polyethylene glycol and a polyamino compound, wherein the aldehyde group and the multi-arm star polyethylene glycol are linked by a chemical bond such as an ether bond, an amide bond, an ester bond, a urethane bond, an imine bond, or a urea bond, and the molar ratio of the amino in the polyamino compound to the aldehyde group in the aldehyde-terminated multi-arm star polyethylene glycol is 0.4-4.4:1, and the polyamino compound is polylysine or a mixture of polylysine and polyethylenimine with a molar ratio of 2-30:3.

The aldehyde-terminated multi-arm polyethylene glycol is a multi-arm polyethylene glycol with not less than 2 arms and a molecular weight of greater than 2000.

The aldehyde-terminated multi-arm polyethylene glycol has 2-8 arms, and preferably 8 arms.

The aldehyde group is selected from one or more of aromatic aldehydes and alkyl aldehydes, and preferably benzaldehyde.

Another object of the present invention is to provide the use of the medical hydrogel in the radiation protection materials, which may be used for preparing radiotherapy spacers, postoperative tissue closure and anti-leakage, anti-tissue adhesion, tissue filler, tissue repair, skin dressing, and pharmaceutical preparation.

A method for preparing a medical hydrogel is provided, comprising dissolving an aldehyde-terminated multi-arm star polyethylene glycol in a pH 4-10 buffer to prepare an aldehyde-terminated multi-arm star polyethylene glycol solution; dissolving a polyamino compound in a pH 4-10 buffer to prepare a polyamino compound solution; and mixing the two solutions to obtain the medical hydrogel.

The aldehyde-terminated multi-arm star polyethylene glycol used in the present invention is commercially available from XIAMEN SINOPEG BIOTECH CO., LTD.

The pH 4-10 buffer is preferably a phosphate buffer or borate buffer with pH 4-10.

The aldehyde-terminated multi-arm star polyethylene glycol solution has a final concentration of 2-30% (w/v), and preferably 10-20% (w/v). The polyamino compound solution has a concentration of 0.5-20%, and preferably 1-5% (w/v).

In the specific use of the present invention, a two-component hydrogel is first prepared, which includes a first component containing nucleophilic functional groups and a second component containing electrophilic functional groups. The first component is an aldehyde-terminated hydrophilic compound with not less than two arms. The hydrophilic compound is an aldehyde-terminated multi-arm star polyethylene glycol, and preferably an eight-arm polyethylene glycol (with a molecular weight of 5000-20000). The aldehyde group is one or more of aromatic aldehydes and alkyl aldehydes, and preferably benzaldehyde. The aldehyde group and the polymer may be linked by a chemical bond that is not easy to hydrolyze, such as an ether bond and an amide bond.

The second component may be a polyamino compound, including one or a mixed component of polylysine (including ε-polylysine and poly-L-lysine) and polyethylenimine.

An amide bond-linked benzaldehyde-terminated eight-arm polyethylene glycol has a structure as follows.

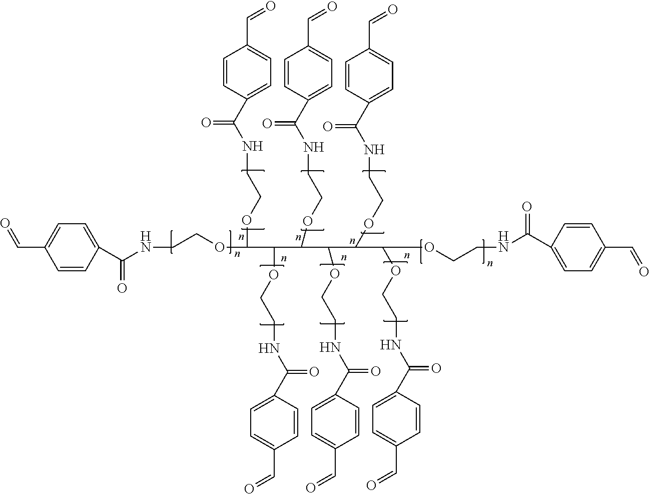

An ether bond-linked benzaldehyde-terminated eight-arm polyethylene glycol has a structure as follows.

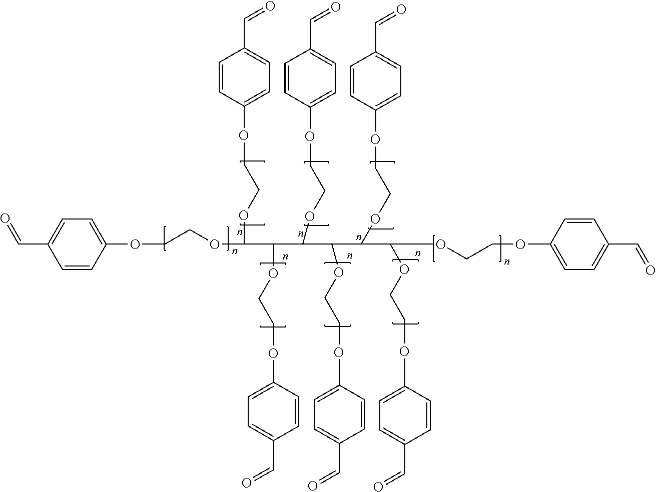

An ester bond-linked benzaldehyde-terminated eight-arm polyethylene glycol has a structure as follows.

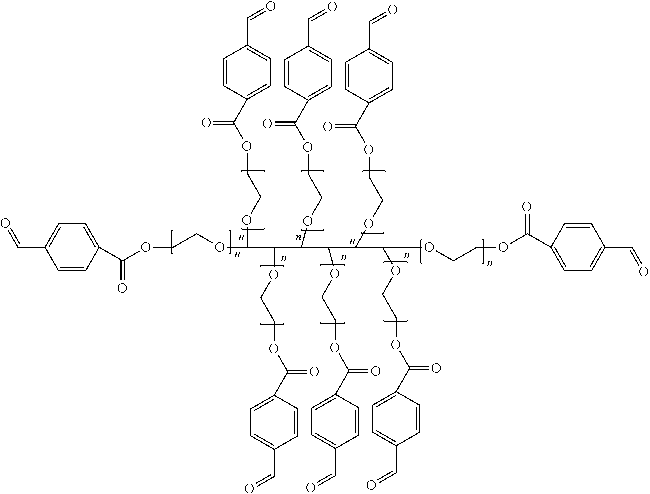

An ether bond-linked propionaldehyde-terminated eight-arm polyethylene glycol has a structure as follows.

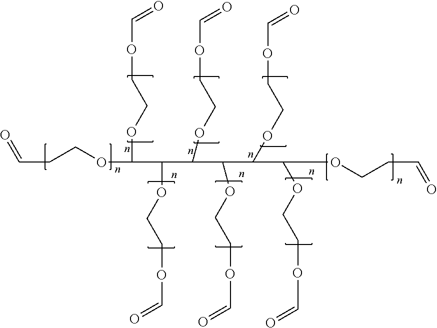

Polylysine and polyethylenimine have the structures as follows, respectively.

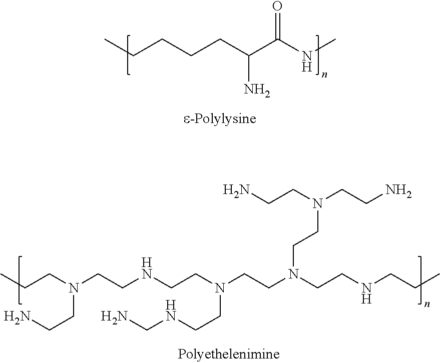

Due to the stability of the aldehyde and amino groups in an aqueous solution, the foregoing two components may be provided in the form of aqueous solution or powder. When in use, the two components are separately dissolved in a buffer, and then the components are mixed to obtain the hydrogel. Alternatively, the two components of the hydrogel may be separately stored in a double-barrel syringe, and when in use, the two components are sprayed or injected to a designated site through a mixing head to form a gel.

In the present invention, the aldehyde group at the end of the multi-arm polyethylene glycol reacts with the amino group in the polyamino compound to produce Schiff base for crosslinking, so that the medical injectable gel is formed.

### Advantages of the Present Invention

The types and proportions of the aldehyde-terminated multi-arm star polyethylene glycol and the polyamino compound in the medical hydrogel are studied in the present invention. It is found that the content of polylysine significantly affects the stability of the hydrogel in an aqueous solution, and when the molar ratio of the amino of polylysine to the aldehyde group of the aldehyde-terminated multi-arm star polyethylene glycol is greater than or equal to 0.4, the hydrogel has a good stability. Moreover, the content of polyethylenimine significantly affects the gelling speed, and when the molar ratio of the amino of polyethylenimine to the aldehyde group of aldehyde-terminated multi-arm star polyethylene glycol is greater than or equal to 0.4, the gelling time is relatively short. The hydrogel obtained by selecting appropriate aldehyde-terminated multi-arm star polyethylene glycol and polyamino compound is fast in gelling, and has a long-term stability in an aqueous solution.

The radiation experiment results of the hydrogel show that the hydrogel still has good swelling property and stability after radiation.

### BRIEF DESCRIPTION OF THE DRAWINGS

FIG. 1 is the observation results of the radiation stability of the hydrogel of the present invention; and

FIG. 2 shows the hydrogel used as a spacer between uterus and surrounding tissues.

### DETAILED DESCRIPTION

The specific steps of the present invention are described by the following examples, but are not limited to the examples.

The terms used in the present invention, unless otherwise stated, generally have the meanings commonly understood by those of ordinary skill in the art.

The present invention is further described below in detail with reference to specific examples and relevant data. It should be understood that the examples are only used to exemplify the present invention, but do not limit the scope of the present invention in any manner.

In the following examples, various processes and methods that are not described in detail are conventional methods known in the art.

The present invention is further described below with reference to specific examples, but the protection scope of the present invention is not limited to this.

### Example 1. Gelling Time and Stability in an Aqueous Solution of the Hydrogels with Different Compositions

600 mg of an amide bond-linked benzaldehyde-terminated eight-arm polyethylene glycol (8-PEG-amide-BA, M.W. 13.5K) was dissolved in 2 mL of phosphate buffer (pH 7.4) to afford solution A. A solution of polylysine and polyethylenimine (M.W. 1.8K) with different contents in phosphate buffer was prepared as solution B. The solution A and the solution B were mixed in equal volume to obtain a viscous hydrogel. The ratios of the amounts of polyethylenimine and polylysine in Table 1 are expressed as the molar ratios of their respective amino groups to the aldehyde group in the aldehyde-terminated multi-arm star polyethylene glycol.

TABLE 1

| | Molar | Molar ratios | | |
|---|---|---|---|---|
| | ratios | of amino in | | |
| | of amino in | polylysine | | |
| | polyethylenimine | to aldehyde | | |
| | to aldehyde | group in | Gelling | |
| | group in 8-PEG- | 8-PEG-amide- | time | Degradation |
| Formulation | amide-BA | BA | (s) | time |
| 1 | 1 | 0 | 2 | less than 48 h |
| 2 | 0.8 | 0.2 | 2 | less than 24 h |
| 3 | 0.6 | 0.4 | 3 | more than 1 |
| | | | | month |
| 4 | 0.4 | 0.4 | 6 | more than 1 |
| | | | | month |
| 5 | 0.4 | 0.6 | 5 | more than 1 |
| | | | | month |
| 6 | 0.4 | 1 | 5 | more than 1 |
| | | | | month |
| 7 | 0.4 | 2 | 5 | more than 1 |
| | | | | month |
| 8 | 0.4 | 3 | 5 | more than 1 |
| | | | | month |
| 9 | 0.4 | 4 | 5 | more than 1 |
| | | | | month |
| 10 | 0.2 | 1 | 20 | more than 1 |
| | | | | month |
| 11 | 0 | 0.4 | 35 | more than 1 |
| | | | | month |
| 12 | 0 | 1 | 25 | more than 1 |
| | | | | month |

The results show that the content of polylysine significantly affects the stability of the hydrogel in an aqueous solution, and when the molar ratio of the amino of polylysine to the aldehyde group of 8-PEG-amide-BA is greater than or equal to 0.4, the hydrogel can remain stable for more than one month. In addition, the content of polyethylenimine significantly affects the gelling speed, and when the molar ratio of the amino of polyethylenimine to the aldehyde group of 8-PEG-amide-BA is greater than or equal to 0.4, the gelling time is relatively short.

400 mg of an ester bond-linked benzaldehyde-terminated eight-arm polyethylene glycol (8-PEG-ester-BA, M.W. 10K) was dissolved in 2 mL of phosphate buffer (pH 7.4) to afford solution A. A solution of polylysine and polyethylenimine (M.W. 1.8K) with different contents in phosphate buffer was prepared as solution B. The solution A and the solution B were mixed in equal volume to obtain a viscous hydrogel. The ratios of the amounts of polyethylenimine and polylysine in Table 2 are expressed as the molar ratios of their respective amino groups to the aldehyde group of the aldehyde-terminated multi-arm star polyethylene glycol.

TABLE 2

| | Molar ratios | Molar ratios | | |
|---|---|---|---|---|
| | of amino in | of amino in | | |
| | polyethylenimine | polylysine | | |
| | to aldehyde | to aldehyde | Gelling | |
| | group in 8-PEG- | group in 8-PEG- | time | Degradation |
| Formulation | ester-BA | ester-BA | (s) | time |
| 1 | 1 | 0 | 3 | less than 48 h |
| 2 | 0.8 | 0.2 | 5 | less than 24 h |
| 3 | 0.6 | 0.4 | 10 | more than 1 |
| | | | | month |
| 4 | 0.4 | 0.6 | 14 | more than 1 |
| | | | | month |
| 5 | 0.4 | 1 | 14 | more than 1 |
| | | | | month |
| 6 | 0.4 | 2 | 13 | more than 1 |
| | | | | month |
| 7 | 0.4 | 3 | 15 | more than 1 |
| | | | | month |

The results show that the content of polylysine significantly affects the stability of the hydrogel in an aqueous solution, and when the molar ratio of the amino of polylysine to the aldehyde group of 8-PEG-ester-BA is greater than or equal to 0.4, the hydrogel can remain stable for more than one month.

### Example 2

400 mg of an amide bond-linked benzaldehyde-terminated eight-arm polyethylene glycol (8-PEG-amide-BA, M.W. 10K) was dissolved in 2 mL of phosphate buffer (pH 7.4) to afford solution A. A solution of 2.44% (w/v) of polylysine (the ratio of amino to aldehyde group is 1:1) in borate buffer was prepared as solution B. The solution A and the solution B were mixed in equal volume to obtain a viscous hydrogel, which has a gelling time of 25 seconds, and can remain stable in vitro for more than 1 month.

### Example 3

600 mg of an amide bond-linked benzaldehyde-terminated eight-arm polyethylene glycol (8-PEG-amide-BA, M.W. 13.5K) was dissolved in 2 mL of phosphate buffer (pH 7.4) to afford solution A. A solution of 1.1% (w/v) of polylysine (the ratio of amino to aldehyde group is 0.4:1) in phosphate buffer was prepared as solution B. The solution A and the solution B were mixed in equal volume to obtain a viscous hydrogel, which has a gelling time of 35 seconds, and can remain stable in vitro for more than 1 month.

### Example 4

600 mg of an amide bond-linked benzaldehyde-terminated eight-arm polyethylene glycol (8-PEG-amide-BA, M.W. 13.5K) was dissolved in 2 mL of phosphate buffer (pH 7.4) to afford solution A. A solution of 2.71% (w/v) of polylysine (the ratio of amino to aldehyde group is 1:1) in phosphate buffer was prepared as solution B. The solution A and the solution B were mixed in equal volume to obtain a viscous hydrogel, which has a gelling time of 22 seconds, and can remain stable in vitro for more than 1 month.

### Example 5

600 mg of an ether bond-linked benzaldehyde-terminated eight-arm polyethylene glycol (8-PEG-amide-BA, M.W. 13.5K) was dissolved in 2 mL of phosphate buffer (pH 7.4) to afford solution A. A solution containing 2.75% (w/v) of polylysine (the ratio of amino to aldehyde group is 1:1) and 0.67% (w/v) of polyethylenimine (M.W. 1.8K) (the ratio of amino to aldehyde group is 0.4:1) in phosphate buffer was prepared as solution B. The solution A and the solution B were mixed in equal volume to obtain a viscous hydrogel, which has a gelling time of 5 seconds, and can remain stable in vitro for more than 1 month.

### Example 6

600 mg of an ether bond-linked benzaldehyde-terminated eight-arm polyethylene glycol (8-PEG-amide-BA, M.W. 13.5K) was dissolved in 2 mL of phosphate buffer (pH 7.4) to afford solution A. A solution containing 8.25% (w/v) of polylysine (the ratio of amino to aldehyde group is 3:1) and 0.67% (w/v) of polyethylenimine (M.W. 1.8K) (the ratio of amino to aldehyde group is 0.4:1) in phosphate buffer was prepared as solution B. The solution A and the solution B were mixed in equal volume to obtain a viscous hydrogel, which has a gelling time of 5 seconds, and can remain stable in vitro for more than 1 month.

### Example 7

400 mg of an ester bond-linked benzaldehyde-terminated eight-arm polyethylene glycol (8-PEG-ester-BA, M.W. 10K) was dissolved in 2 mL of phosphate buffer (pH 7.4) to afford solution A. A solution containing 2.44% (w/v) of polylysine and 0.59% (w/v) of polyethylenimine (M.W. 1.8K) in borate buffer was prepared as solution B. The solution A and the solution B were mixed in equal volume to obtain a viscous hydrogel, which has a gelling time of 15 seconds, and can remain stable in vitro for more than 1 month.

### Example 8. Radiation Stability of Hydrogel

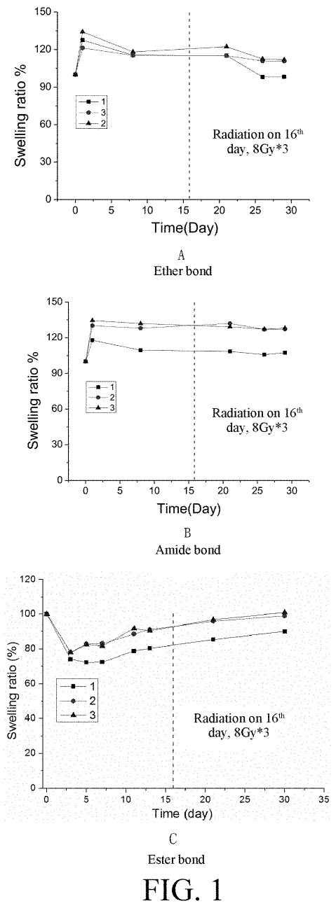

600 mg of an amide bond/ether bond-linked benzaldehyde-terminated eight-arm polyethylene glycol (8-PEG-amide-BA, M.W. 13.5K) was dissolved in 2 mL of phosphate buffer (pH 7.4) to afford solution A. A solution containing 2.75% (w/v) of polylysine and 1.1% (w/v) of polyethylenimine (M.W. 1.8K) in borate buffer was prepared as solution B. 400 mg of an ester bond-linked benzaldehyde-terminated eight-arm polyethylene glycol (8-PEG-ester-BA, M.W. 10K) was dissolved in 2 mL of phosphate buffer (pH 7.4) to afford solution A. A solution containing 2.44% (w/v) of polylysine and 0.59% (w/v) of polyethylenimine (M.W. 1.8K) in borate buffer was prepared as solution B. The solution A and the solution B were mixed in equal volume to obtain a viscous hydrogel. The samples were prepared in triplicate, wherein sample 1 was not radiated, and the sample 1 and sample 2 were parallel samples. The sample 1 and sample 2 hydrogels were placed in PBS solution at 37° C. for 16 days. On the 16^{th} day, the hydrogels were subject to three 8Gy radiations, and then were placed in PBS solution at 37° C. again. The experiment results are shown in FIG. 1 (FIG. 1A, FIG. 1B, and FIG. 1C respectively correspond to the observation results of the radiation stabilities of the hydrogels having an aldehyde group and a multi-arm star polyethylene glycol linked through an ether bond, an amide bond, and an ester bond). The results indicate that the hydrogel of the present invention has good radiation stability.

### Example 9. Use of Hydrogel as a Spacer Between Uterus and Surrounding Tissues

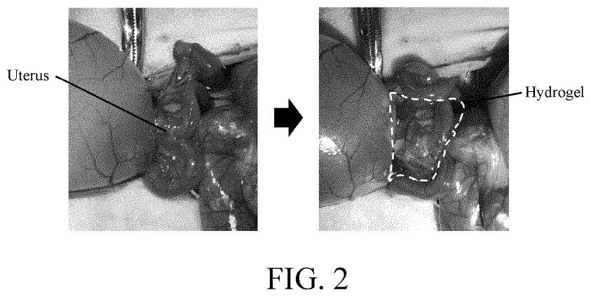

The hydrogel solution A and hydrogel solution B of the present invention were mixed and injected around the uterus of a female rabbit with a mixed syringe. Once the mixed solution reached the outer wall of the uterus, it underwent a gelling reaction, and adhered to the outer wall of the uterus. Results are shown in FIG. 2. The results show that the hydrogel forms a physical barrier between the uterus and other organs (e.g., rectum and bladder), which can decrease the injuries to adjacent tissues during radiotherapy of uterus tumors.

1. A medical hydrogel, formed by in-situ crosslinking an aldehyde-terminated multi-arm star polyethylene glycol and a polyamino compound, wherein the aldehyde group and the multi-arm star polyethylene glycol are linked by a chemical bond such as an ether bond, an amide bond, an ester bond, a urethane bond, an imine bond, or a urea bond, and the molar ratio of the amino in the polyamino compound to the aldehyde group in the aldehyde-terminated multi-arm star polyethylene glycol is 0.4-4.4:1, and the polyamino compound is polylysine or a mixture of polylysine and polyethylenimine in a molar ratio of 2-30:3.

2. The medical hydrogel according to claim 1, wherein the aldehyde-terminated multi-arm star polyethylene glycol is a multi-arm polyethylene glycol with more than 2 arms and a molecular weight of greater than 2000.

3. The medical hydrogel according to claim 1, wherein the aldehyde-terminated multi-arm star polyethylene glycol has 2-8 arms.

4. The medical hydrogel according to claim 1, wherein the aldehyde group is selected from one or more of aromatic aldehydes and alkyl aldehydes.

5. Use of the medical hydrogel according to claim 1 in the preparation of a radiation protection material.

6. A method for preparing the medical hydrogel according to claim 1, comprising: dissolving the aldehyde-terminated multi-arm star polyethylene glycol in a pH 4-10 buffer to prepare an aldehyde-terminated multi-arm star polyethylene glycol solution; dissolving the polyamino compound in a pH 4-10 buffer to prepare a polyamino compound solution; and mixing the two solutions to obtain the medical hydrogel.

7. The method for preparing the medical hydrogel according to claim 6, wherein the aldehyde-terminated multi-arm star polyethylene glycol solution has a final concentration of 2-30%, and the polyamino compound solution has a concentration of 0.5-20%.

8. The method for preparing the medical hydrogel according to claim 6, wherein the aldehyde-terminated multi-arm star polyethylene glycol solution has a final concentration of 10-20%, and the polyamino compound solution has a concentration of 1-5%.
